(19) United States
(12) Patent Application Publication
(10) Pub. No.: US 2008/0044833 A1
(43) Pub. Date: Feb. 21, 2008

(54) Diagnostic PKM2 methods and compositions

(76) Inventor: Timothy D. Connors, Shrewsbury, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/803,960

(22) Filed: May 16, 2007

Related U.S. Application Data
(63) Continuation of application No. PCT/US05/42074, filed on Nov. 15, 2005.
(63) Continuation of application No. PCT/US05/43590, filed on Dec. 1, 2005.
(60) Provisional application No. 60/628,569, filed on Nov. 16, 2004.

(51) Int. Cl. C12Q 1/68; C12Q 1/48; G01N 33/573

(52) U.S. Cl. 435/6; 435/15; 435/7.4

(57) ABSTRACT

This invention provides a method for diagnosing or prognosing ovarian cancer by detecting the level of expression of pyruvate kinase isoenzyme M2 or fragment thereof in a subject. Applicants report that overexpression of this protein or fragment thereof is correlative with the presence of ovarian cancer in mammals. Applicants also report that overexpression of the protein is not correlative with the presence of an immune response, e.g., the generation of anti-PKM2 antibodies detected by Western blot analysis. Also provided are diagnostic kits and methods to assay for new candidate drugs that treat or ameliorate the symptoms associated with ovarian cancer.

### CROSS-REFERENCE TO RELATED APPLICATIONS

This application claims priority under 35 U.S.C. §119(e) to U.S. Provisional Application, U.S. Ser. No. 60/628,569 filed Nov. 16, 2004. The content of this application is incorporated by reference into the present disclosure.

### TECHNICAL FIELD

The invention relates to methods for screening for human cancers and related malignancies.

### BACKGROUND OF THE INVENTION

Pyruvate-kinase (ATP:pyruvate 2-O-phosphotransferase, PK) is a key enzyme in the glycolytic pathway that catalyses the formation of pyruvate and ATP from phosphoenolpyruvate and ADP. Munoz and Ponce (2003) Comp. Bio. Phys. Part B 135:197-218. In mammals, PK exists as four isoenzymes named as M1-, M2-, L- and R-types, the expression of which differ from one cell type to another. Tanaka et al. (1967) J. Biochem. (Tokyo) 62:71-91; Ibsen et al. (1974) Arch. Biochem. Biophys. 570-580; and Nowak et al. (1981) Mol. Cell. Biochem. 35:65-75. The M2-type is predominant in the fetus, in neuplasias and in undifferentiated or proliferating tissues, but also widely distributed in adult tissues. Mallati et al. (1992) Cancer Biochem. Biophys. 13:33-41; and Guminska et al. (1988) Biochem. Biophys. Acta 966:207-213. M2-type is progressively replaced by the M1-type in skeletal muscle, heart and brain during differentiation. The M1- and M2-type are produced from the same gene by alternative splicing. Takenaka et al. (1991) J. Biochem 198(l):101-106. The difference between these two isoforms is in one exon encoding 51 amino acid residues, in which 21 residues are different. The enzymological property of the M1-type isoenzyme is very different from the other three forms since it is the only one that is allosterically not regulated. The M1-type shows hyperbolic kinetics and is not activated by fructose-1,6-bisphosphate (FBP) in contrast, M2-type shows kinetic curve and is activated by FBP. Saheki et al. (1982) Biochem. Biophys. Acta 704:484-493.

PK has been used as a tumor marker, as the isoform M2 isolated from patient samples is shown to be strongly overexpressed in hyperproliferative diseases. Oremek et al. (2003) Anticancer Res. 23(2A):1131-1134 reports that plasma concentrations of tumor PKM2 were increased in patients with rheumatoid diseases such as rheumatoid arthritis, systemic lupus erythematosus and seronegative spondylarthritis. In a separate publication, the authors also report overexpression in patients suffering from haematological malignancies such as chronic myelocytic leukaemia, chronic lymphocytic leukaemia and lymphocytic leukaemia. Oremek et al. (2003) Anticancer Res. 23(2A): 1135-1138. Others have suggested its use as a tumor marker for lung cancer (Schneider et al. (2003) Anticancer Res. 23(2A):899-906) gastric cancer, colorectal cancer, colorectal adenomas (Schneider and Schulze (2003) Anticancer Res. 23(6D):5089-5093; Hardt et al. (2003) Anticancer Res. 23(2A):851-853 and 23(2A):851-853 and 23(2A):855-857; Hardt et al. (2004) Br. J. Cancer 91(5):980-984), pancreatic cancer (Ventrucci et al. (2004) Dig. Dis. Sci. 49(7-8):1149-1155), cervical carcinoma (Kaura et al. (2004) J. Obstet. Gyn. Res. 30(3):193-196) and breast cancer (Luftner et al. (2003) Anticancer Res. 23(2A):991-997). Commercial use of the PKM2 isoenzyme as a diagnostic aid has been disclosed in U.S. Patent Publication Nos. US 2004/0152648A1 and US 2002/0102623A1.

It also has been assayed in other cancers and not recommended as a marker for metastatic renal carcinoma (Roigas et al. (2003) Urol. Res. 31(6):358-362), neuroendocrine tumors (Pezzilli et al. (2003) Anticancer Res. 23(3C): 2969-2972), diabetic nephropathy (Oremek et al. (2003) Anticancer Res. 23(2A): 1155-1158), Barrett's adenocarcinoma (Koss et al. (2004) J. Clin. Pathol. 57(11):1156-1159) and malignant thyroid disease (Bena-Boupda et al. (2003) Anticancer Res. 23(6D):5237-5240.

### DISCLOSURE OF THE INVENTION

This invention provides a method for diagnosing or prognosing ovarian cancer by detecting the level of expression of pyruvate kinase isoenzyme M2 or fragment thereof in a subject. Applicants report that overexpression of this protein or fragment thereof is correlative with the presence of ovarian cancer in mammals. Applicants also report that overexpression of the protein is not correlative with the presence of an immune response, e.g., the generation of anti-PKM2 antibodies detected by Western blot analysis.

Also provided are diagnostic kits and methods to assay for new candidate drugs that treat or ameliorate the symptoms associated with ovarian cancer.

### BRIEF DESCRIPTION OF THE FIGURES

FIG. 1 shows a PKM2 ELISA standard curve.

FIG. 2 shows the results of the PKM2 ELISA versus ovarian patient serum samples.

### MODES OF CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature, see for example, the following publications, Sambrook et al. MOLECULAR CLONING: A LABORATORY MANUAL, 2^{nd} edition (1989); CURRENT PROTOCOLS IN MOLECULAR BIOLOGY (F. M. Ausubel et al. eds. (1987)); the series METHODS IN ENZYMOLOGY (Academic Press, Inc.); PCR: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1988)); USING ANTIBODIES, A LABORATORY MANUAL (Harlow and Lane eds. (1999)); and ANIMAL CELL CULTURE (R. I. Freshney ed. (1987)).

Definitions

As used herein, certain terms may have the following defined meanings.

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

The term “antigen” is well understood in the art and includes substances which are immunogenic. The term as used herein also includes substances which induce immunological unresponsiveness or anergy.

A “native” or “natural” or “wild-type” antigen is a polypeptide, protein or a fragment which contains an epitope and which has been isolated from a natural biological source. It also can specifically bind to an antigen receptor.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention.

The antibodies can be polyclonal or monoclonal and can be isolated from any suitable biological source, e.g., murine, rat, sheep and canine. Additional sources are identified infra.

The term “pyruvate-kinase” as used herein means pyruvate-kinases selected from isoenzymes M1, M2, L and R, as well as other isoenzymes. Particularly, the pyruvate-kinase is isoenzyme isoenzyme M2 (GenBank Accession No. M23725.1 and Swiss-Prot. number 14786). The DNA sequence encoding pyruvate kinase isoform M1 and M2 is disclosed in GenBank Accession number X56494. M2 protein is provided under GenBank Accession No. M23725.1. Pyruvate-kinases are obtainable from human or non-human animal sources, particularly from mammalian sources, such as man, mouse, rat, hamster, monkey, pig, etc. Sequence comparison is provided by Munoz and Ponce (2003) supra. Especially preferred is the human M2-pyruvate-kinase comprising:

a) the amino acid sequence as shown in under GenBank Accession No. M23725.1 or Swiss-Prot. number P14786; or

b) an amino acid sequence having an identity of at least 80%, particularly of at least 90% and more particularly of at least 95% thereto, wherein the amino acid sequence identity may be determined by a suitable computer-program, such as GCG or BLAST.

Furthermore, the term “pyruvate-kinase” encompasses recombinant derivatives or variants thereof, as well as fragments thereof having biological activity of isoform M2, for example. These derivatives, variants and fragments thereof may be obtained as expression products from allelic variant genes or from recombinantly altered ones, e.g. modified or truncated genes and/or as products of protolytic cleavage.

The pyruvate-kinase protein is encoded by a nucleic acid which may be a DNA or an RNA. Preferably, the nucleic acid comprises:

a) the nucleic acid sequence as shown in GenBank Accession number M23725.1 or X56494 or complementary thereto;

b) a nucleic acid sequence corresponding to the sequence corresponding to (a) within the scope of degeneracy of the genetic code; or

c) a nucleic acid sequence hybridizing under stringent conditions with a sequence of (a) and/or (b).

An “anti-PKM2 antibody” of this invention is an antibody that selectively binds pyruvate kinase isoform M2 protein. Anti-PKM2 antibodies are commercially available and methods for making them and the hybridoma cell lines which produce them are described infra.

The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′)2 fragment, a bivalent fragment comprising two Fab fragments linked by a disulfide bridge at the hinge region; a Fd fragment consisting of the VH and CH, domains; a Fv fragment consisting of the VL and VH domains of a single arm of an antibody, a dAb fragment (Ward et al. (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl. Acad Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “antibody” variant is intended to include antibodies produced in a species other than a mouse or an isotype of an antibody of this invention. The term “antibody variant” also includes antibodies containing post-translational modifications to the linear polypeptide sequence of the antibody or fragment. It further encompasses fully human antibodies.

The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (V_{L}, V_{H})) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or I amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

A “human monoclonal antibody” refers to antibodies displaying a single binding specificity which have variable and constant regions derived from human germline immunoglobulin sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM or IgG1) that is encoded by heavy chain constant region genes.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-stranded, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. In addition to a native nucleic acid molecule, a nucleic acid molecule of the present invention may also comprise modified nucleic acid molecules.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein.

The term “genetically modified” means containing and/or expressing a foreign gene or nucleic acid sequence which in turn, modifies the genotype or phenotype of the cell or its progeny. In other words, it refers to any addition, deletion or disruption to a cell's endogenous nucleotides.

As used herein, “expression” refers to the process by which polynucleotides are transcribed into mRNA and translated into peptides, polypeptides, or proteins. If the polynucleotide is derived from genomic DNA of an appropriate eukaryotic host expression may include splicing of the mRNA. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods known in the art, for example, the methods herein below for constructing vectors in general.

“Under transcriptional control” is a term understood in the art and indicates that transcription of a polynucleotide sequence, usually a DNA sequence, depends on its being operatively linked to an element which contributes to the initiation of, or promotes, transcription. “Operatively linked” refers to a juxtaposition wherein the elements are in an arrangement allowing them to function.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide can be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Zaks et al. (1999) Nat. Med. 7:823-827. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, “retroviral vector” refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad), pseudo adenoviral or adeno-associated virus (AAV), vector construct refers to the polynucleotide comprising the viral genome or part thereof, and a transgene. Adenoviruses (Ads) are a relatively well characterized, homogenous group of viruses, including over 50 serotypes. See, e.g., WO 95/27071. Ads are easy to grow and do not require integration into the host cell genome. Recombinant Ad-derived vectors, particularly those that reduce the potential for recombination and generation of wild-type virus, have also been constructed. See, WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, nucleic acids or proteins of this invention can be conjugated to antibodies or binding fragments thereof which bind cell surface antigens, e.g., TCR, CD3 or CD4.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art. Preferably, default parameters are used for alignment. One alignment program is BLAST, using default parameters. Alternative programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases =non-redundant, GenBank+EMBL+DDBJ+PDB+GenBank CDS translations+SwissProtein+SPupdate+PIR. Details of these programs can be found at the following Internet address: ncbi.nim.nih.gov/cgi-bin/BLAST.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated”, “separated” or “diluted” polynucleotide, peptide, polypeptide, protein, antibody, or fragment(s) thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragment(s) thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as its glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

“Host cell,” “target cell” or “recipient cell” are intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human.

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

As used herein, the terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures.

Tumor cell growth can be assessed by any means known in the art, including, but not limited to, measuring tumor size, determining whether tumor cells are proliferating using a ³H-thymidine incorporation assay, or counting tumor cells. Hyperplasia is a form of controlled cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. Atypical metaplasia involves a somewhat disorderly metaplastic epithelium.

A “composition” is intended to mean a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

An additional example of “carriers” includes therapeutically active agents such as another peptide or protein (e.g., an Fab′ fragment). For example, an antibody of this invention, variant, derivative or fragment thereof can be functionally linked (e.g., by chemical coupling, genetic fusion, noncovalent association or otherwise) to one or more other molecular entities, such as another antibody (e.g., to produce a bispecific or a multispecific antibody), a cytotoxin, a cellular ligand or an antigen. Accordingly, this invention encompasses a large variety of antibody conjugates, bi- and multispecific molecules, and fusion proteins, whether or not they target the same epitope as the antibodies of this invention.

Yet additional examples of carriers are organic molecules (also termed modifying agents) or activating agents, that can be covalently attached, directly or indirectly, to an antibody of this invention. Attachment of the molecule can improve pharmacokinetic properties (e.g., increased in vivo serum half-life). Examples of organic molecules include, but are not limited to a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane.

Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. A suitable hydrophilic polymer that modifies the antibody of the invention has a molecular weight of about 800 to about 150,000 Daltons as a separate molecular entity. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Examples of such include, but are not limited to n-dodecanoate, n-tetradecanoate, n-octadecanoate, n-eicosanoate, n-docosanoate, n-triacontanoate, n-tetracontanoate, cis-.DELTA.9-octadecanoate, all cis-.DELTA.5,8,11,14-eicosatetraenoate, octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

An “effective amount” is an amount sufficient to effect beneficial or desired results. An effective amount can be administered in one or more administrations, applications or dosages.

This invention provides a method for diagnosing or prognosing ovarian cancer in a subject by detecting the level of expression of pyruvate kinase isoenzyme M2 or fragment thereof in the subject or a sample isolated from the subject. In one aspect, the subject is not pregnant.

The samples include, but are not limited to ovarian tissue or a body fluid, e.g., serum sample, blood sample or urine sample. Overexpression of the protein or fragment thereof in an amount of at least 1.2× normal, or alternatively at least 1.5× normal, or alternatively, at least 2.0× normal, or alternatively, at least 2.5× normal, is indicative of a predisoposition or diagnosis of ovarian cancer in the subject such as mammal or human.

In one aspect, the level of expression of pyruvate kinase isoenzyme or fragment thereof is determined immunochemically using an anti-PKM2 specific antibody. In one aspect, more than one (e.g., at least two) specific antibodies that can only bind to tumor PKM2 are used in order to possibly achieve an even higher specificity. In yet a further aspect, the antibody or antibodies are monoclonal antibodies that specifically bind to tumor PKM2 and discriminates between other pyruvate kinase isoenzymes such as PKM1. This in one embodiment, the antibody is substantially non-reactive with any pyruvate kinase isoenzyme (i.e., other than PKM2).

Anti-PKM2 monoclonal antibodies are commercially available or can be generated using techniques known in the art and described herein. The amino acid and polynucleotide sequences are publicly available and can be used to reproduce protein or a immunogenic fragment thereof and used in the methods to produce PKM2 specific monoclonal and polyclonal antibodies. Variants, derivatives and humanized antibodies can be used in this methods. Methods to produce these are described herein.

In one aspect, the anti-PKM2 antibody is labeled to facilitate detection of any PKM2 protein/antibody conjugate produced during performance of a method of this invention.

In practicing this method, the antibody which binds specifically to PKM2 is immobilized on a solid phase by means of known methods. The solid support can also be included in any test kit. A suitable sample suspected of cotaining PKM2 protein or a fragment thereof is is then contacted with the preferably dissolved protein and bound to the solid phase in a suitable buffer system by means of the antibodies. After washing the immobilized antibody-PKM2 complex obtained in this manner, a further labelled second antibody (e.g. carrying biotin) is then added which binds to another epitope of PKM2. Detection of the labe can be achieved by any method known in the art, e.g., with the aid of streptavidin-peroxidase. In a further aspect, the the test kit contains reference material with a known content of tumor PKM2 as a control.

It is also possible to perform the method using biosensors such as amperometric sensors, potentiometric, ion-selective potentiometric or photometric sensors or those employing semiconductor electrodes such as field effect transistors (FET), chemosensitive field effect transistors (CHEMFET), suspended-gate field effect transistors (SGFET) or ion-sensitive field effect transistors. See E. A. H. Hall and G. Hummel in “BIOSENSOPEN”, Springer Verlag Heidelberg, Germany (1995). Further developments of ion-sensitive field effect transistors (ISFET) or optical detectors are known in the art. For example, the method of the invention can be performed using piezoelectric oscillating quartz crystals and surface wave elements that can be used as microbalances. In this aspect, the primary antibody (the so-called catcher) is immobilized on a piezoelectric substrate and a change in the oscillating frequency of the quartz crystal is measured after binding to the pyruvate kinase isoenzyme of the type tumor M2-PK to be analysed. Such sensors are described for example by A. Leidl et al., in “PROCEEDINGS OF THE SECOND INTERNATIONAL SYMPOSIUM ON MINIATURIZED TOTAL ANALYSES SYSTEMS .MU.TAS”, Basle (1996). Quartz crystal microbalances as described by C. Koslinger et al., (1994) J. Anal. Chem 349:349-354 can be used.

Alternatively, it is also possible to determine PKM2 concentration using immunochromatographic methods such as so-called visual rapid tests or lateral flow tests.

Another method according to the invention is a method for immunohistochemical detection of any of the peptides of the invention in tissue sections, using, as the detecting antibody, any monoclonal antibody which reacts with one of the peptides of the invention, or any polyclonal antibody which specifically binds pyruvate-kinase M2 isoform.

The present invention further provides a method for diagnosing ovarian cancer in a cell, tissue, organ, animal or patient and/or, prior to, subsequent to, or during the course of the disease. Additionally, one can assay for candidate agents to lower the expression level of PKM2 and therefore treat ovarian cancer by assaying for the level of PKM2 in a sample isolated from the subject before, during and after treatment.

The invention also provides an article of manufacture, comprising packaging material and at least one vial comprising a solution of an PKM2 diagnostic agent, e.g., anti-PKM2 antibody or nucleic acid probe or chip, with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater.

In another aspect, the level of PKM2 expressison can be determined by determining the amount of PKM2 mRNA in the sample using PKM2-specific probes, primers or chips containing one or more of probes or primers. Methods for determining the level of polynucleotide expression are known in the art and described below.

Further provided by the present invention are methods for aiding in the detecting, diagnosing, prognosing, and monitoring the progression, course, or stage of ovarian cancers or ovarian malignancies in subjects afflicted therewith. These invention methods comprise detecting the differential expression of PKM2 in a sample isolated from a cell or tissue, wherein the presence and/or amount of the PKM2 is indicative of the neoplastic condition of cell or tissue. A related neoplasia is one in which the expression or expression of the protein serves as a marker for the ovarian cancer or overian neoplasia. Samples of cells or tissue can be provided free form or attached to a solid support and can be isolated from a tissue culture, commercially available cell line, from a patient biopsy or as in the case of use of the method for tissue imaging, in vivo.

Diagnostic kits to practice the methods described herein are also provided. In one aspect, the kit contains at least one agent that specifically recognizes and binds to a PKM2 protein or peptide and a detection reagent which can support a reporter group. When antibodies are used as the agent, they can be in solution or immobilized on a solid support, such as nitrocellulose, glass, latex or a plastic. Examples of detection reagents include but are not limited to anti-immunoglobulin, protein G, protein A, or lectin. Reporter groups can be provided in the kit, but are not necessarily supplied. Examples of reporter groups suitable for use in the methods include but are not limited to radioisotopes, fluorescent groups, luminescent groups, enzymes, biotin and dye particles.

The kits will contain one or more of binding agents described above, e.g., a probe, a primer, an antibody or immune effector cell specific for any of the peptides of the invention, or an agent that specifically recognizes and binds to the specific antibody, e.g., antibodies raised and isolated to specifically recognize and bind PKM2 protein or fragment thereof. Instructions for use can also be provided.

Antibodies

Antibodies useful in the methods of this invention are polyclonal or monoclonal antibodies. They can be chimeric, humanized, or totally human. A functional fragment of an antibody includes but is not limited to Fab, Fab′, Fab2, Fab′2, and single chain variable regions. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. So long as the fragment or derivative retains specificity of binding to PKM2 or a fragment thereof, it can be used. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific. Specific assays, e.g., ELISA, for determining specificity are described infra.

The monoclonal antibodies of the invention can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived therefrom, or any other suitable cell line as known in the art (see, e.g., www.atcc.org, www.lifetech.com., and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al., (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al., (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al., Proc. Natl. Acad. Sci. USA (1996) 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994).

Antibody variants of the present invention can also be prepared using delivering a polynucleotide encoding an antibody of this invention to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody variant” includes post-translational modification to linear polypeptide sequence of the antibody or fragment. For example, U.S. Pat. No. 6,602,684 B I describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody variants also can be prepared by delivering a polynucleotide of this invention to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies of the present invention can also be produced using transgenic plants, according to know methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present invention can be performed using any known method, such as but not limited to those described in U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel, N. D. et al. (2000) Infection and Immunity April 2000:1820-1826; Gallo, M. L. et al. (2000) European J. of Immun. 30:534-540; Green, L. L. (1999) J. of Immun. Methods 231:11-23; Yang, X-D et al. (1999A) J. of Leukocyte Biology 66:401-410; Yang, X-D (1999B) Cancer Research 59(6):1236-1243; Jakobovits, A. (1998) Advanced Drug Delivery Reviews 31:33-42; Green, L. and Jakobovits, A. (1998) J. Exp. Med. 188(3):483-495; Jakobovits, A. (1998) Exp. Opin. Invest. Drugs 7(4):607-614; Tsuda, H. et al. (1997) Genomics 42:413-421; Sherman-Gold, R. (1997). Genetic Engineering News 17(14); Mendez, M. et al. (1997) Nature Genetics 15:146-156; Jakobovits, A. (1996) WEIR'S HANDBOOK OF EXPERIMENTAL IMMUNOLOGY, THE INTEGRATED IMMUNE SYSTEM VOL. IV, 194.1-194.7; Jakobovits, A. (1995) Current Opinion in Biotechnology 6:561-566; Mendez, M. et al. (1995) Genomics 26:294-307; Jakobovits, A. (1994) Current Biology 4(8):761-763; Arbones, M. et al. (1994) Immunity 1(4):247-260; Jakobovits, A. (1993) Nature 362(6417):255-258; Jakobovits, A. et al. (1993) Proc. Natl. Acad. Sci. USA 90(6):2551-2555; Kucherlapati, et al. U.S. Pat. No. 6,075,181.)

Human monoclonal antibodies can also be produced by a hybridoma which includes a B cell obtained from a transgenic nonhuman animal, e.g., a transgenic mouse, having a genome comprising a human heavy chain transgene and a light chain transgene fused to an immortalized cell.

These antibodies can be modified to create chimeric antibodies. Chimeric antibodies are those in which the various domains of the antibodies' heavy and light chains are coded for by DNA from more than one species. See, e.g., U.S. Pat. No. 4,816,567.

The term “antibody derivative” also includes “diabodies” which are small antibody fragments with two antigen-binding sites, wherein fragments comprise a heavy chain variable domain (V_{H}) connected to a light chain variable domain (V_{L}) in the same polypeptide chain (VH V_{L}). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.

The term “antibody derivative” further includes “linear antibodies”. The procedure for making the is known in the art and described in Zapata et al. (1995) Protein Eng. 8(10):1057-1062. Briefly, these antibodies comprise a pair of tandem Fd segments (V_{H}-C_{H} 1-VH-C_{H}1) which form a pair of antigen binding regions. Linear antibodies can be bispecific or monospecific.

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present invention include naturally purified products, products of chemical synthetic procedures, and products produced by recombinant techniques from a eukaryotic host, including, for example, yeast, higher plant, insect and mammalian cells, or alternatively from a prokaryotic cells as described above.

In some aspects of this invention, it will be useful to detectably or therapeutically label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³ Bi), technetium-99m (^{99m}Tc), rhenium-186 (¹⁸⁶Re), and rhenium-188 (¹⁸³Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

With respect to preparations containing antibodies covalently linked to organic molecules, they can be prepared using suitable methods, such as by reaction with one or more modifying agents. Examples of such include modifying and activating groups. A “modifying agent” as the term is used herein, refers to a suitable organic group (e.g., hydrophilic polymer, a fatty acid, a fatty acid ester) that comprises an activating group. Specific examples of these are provided supra. An “activating group” is a chemical moiety or functional group that can, under appropriate conditions, react with a second chemical group thereby forming a covalent bond between the modifying agent and the second chemical group. Examples of such are electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art. See for example, Hermanson, G. T., BIOCONJUGATE TECHNIQUES, Academic Press: San Diego, Calif. (1996). An activating group can be bonded directly to the organic group (e.g., hydrophilic polymer, fatty acid, fatty acid ester), or through a linker moiety, for example a divalent C1-C12 group wherein one or more carbon atoms can be replaced by a heteroatom such as oxygen, nitrogen or sulfur. Suitable linker moieties include, for example, tetraethylene glycol. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl)carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid.

The modified antibodies of the invention can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis. See generally, Hermanson, G. T., BIOCONJUGATE TECHNIQUES, Academic Press: San Diego, Calif. (1996).

Methods for Generating and Delivering Polynucleotides

Certain embodiments of this invention require the use of polynucleotides, for example to recombinantly produce PKM2 protein or for primers or probes. These can be generated and replicated using any method known in the art for example by using commercial DNA synthesizer to replicate the DNA. Alternatively, they can be obtained by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, the polynucleotide can be inserted into a suitable replication vector which is then inserted into a suitable host cell (prokaryotic or eukaryotic) for replication and amplification. The DNA so amplified can be isolated from the cell by well known methods.

RNA can be obtained by first inserting a DNA polynucleotide. The DNA can by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook et al. (1989) supra, such as by using various lytic enzymes or chemical solutions or extracted by nucleic-acid-binding resins.

When the agent is a nucleic acid, it can be added to the cell cultures by methods known in the art, which include, but are not limited to calcium phosphate precipitation, microinjection or electroporation. They can be added alone or in combination with a suitable carrier, e.g., a vector, liposome, or a pharmaceutically acceptable carrier such as phosphate buffered saline. Vectors that contain both a promoter and a cloning site into which a polynucleotide can be operatively linked are known in the art. Such vectors are capable of transcribing RNA in vitro or in vivo, and are commercially available. In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of vectors are viruses, such as baculovirus and retrovirus, bacteriophage, adenovirus, adeno-associated virus, cosmid, plasmid, fungal vectors and other recombination vehicles typically used in the art which have been described for expression in a variety of eukaryotic and prokaryotic hosts. Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens. Liposomes that also comprise a targeting antibody or fragment thereof can be used in the methods of this invention.

Preparation and Isolation of Proteins and Polypeptides

Polypeptides and proteins are necessary components to generate antibodies and markers useful in various methods of this invention. For example, recombinant antibodies, variants, derivatives and fragments thereof can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Alternatively, the proteins and polypeptides can be obtained by known recombinant methods as described herein using the host cell and vector systems described above. They can also be prepared by enzymatic digestion or cleavage of naturally occurring proteins.

Proteins and peptides can be isolated or purified by standard methods including chromatography (e.g., ion exchange, affinity, and sizing column chromatography), centrifugation, differential solubility, or by any other standard technique for protein purification. Alternatively, affinity tags such as hexa-His (Invitrogen), Maltose binding domain (New England Biolabs), influenza coat sequence (Kolodziej et al. (1991) Methods Enzymol. 194:508-509), and glutathione-S-transferase can be attached to the peptides of the invention to allow easy purification by passage over an appropriate affinity column. Isolated peptides can also be physically characterized using such techniques as proteolysis, nuclear magnetic resonance, and x-ray crystallography.

It is well known that modifications can be made to any peptide to provide it with altered properties. Peptides for use in this invention can be modified to include unnatural amino acids. Thus, the peptides may comprise D-amino acids, a combination of D- and L-amino acids, and various “designer” amino acids (e.g., β-methyl amino acids, C-β-methyl amino acids, and N-α-methyl amino acids, etc.) to convey special properties to peptides.

In a further embodiment, subunits of peptides that confer useful chemical and structural properties will be chosen. For example, peptides comprising D-amino acids may be resistant to L-amino acid-specific proteases in vivo. Modified compounds with D-amino acids may be synthesized with the amino acids aligned in reverse order to produce the peptides of the invention as retro-inverso peptides. In addition, the present invention envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂NH—R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide peptides with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present invention provides a method to produce a constrained peptide that incorporates random sequences at all other positions.

Methods to Detect Nucleic Acids

In certain aspects of this invention, it may be necessary to detect and quantifying the expression of PKM2. These methods are generally known in the art and include but are not limited to hybridization assays (Northern blot analysis) and PCR based hybridization assays.

To assay for an alteration in mRNA level using hybridization assays, the nucleic acid contained in a sample is first extracted. For instance, mRNA can be isolated using various lytic enzymes or chemical solutions according to the procedures set forth in Sambrook et al. (1989), supra, or extracted by commercially available nucleic-acid-binding resins following the accompanying instructions provided by the manufacturers. The mRNA contained in the extracted nucleic acid sample is then detected by hybridization (e.g., Northern blot analysis) and/or amplification procedures using nucleic acid probes and/or primers, respectively, according to standard procedures.

Nucleic acid molecules having at least 10 nucleotides and exhibiting sequence complementarity or homology to the nucleic acid to be detected can be used as hybridization probes or primers in the diagnostic methods. It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. The total size of fragment, as well as the size of the complementary stretches, will depend on the intended use or application of the particular nucleic acid segment. Smaller fragments of the gene will generally find use in hybridization embodiments, wherein the length of the complementary region may be varied, such as between about 10 and about 100 nucleotides, or even full length according to the complementary sequences one wishes to detect.

Nucleotide probes having complementary sequences over stretches greater than about 10 nucleotides in length will increase stability and selectivity of the hybrid, and thereby improving the specificity of particular hybrid molecules obtained. One can design nucleic acid molecules having gene-complementary stretches of more than about 25 and even more preferably more than about 50 nucleotides in length, or even longer where desired. Such fragments may be readily prepared by, for example, directly synthesizing the fragment by chemical means, by application of nucleic acid reproduction technology, such as the PCR™ technology with two priming oligonucleotides as described in U.S. Pat. No. 4,603,102 or by introducing selected sequences into recombinant vectors for recombinant production.

In certain embodiments, it will be advantageous to employ nucleic acid sequences of the present invention in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. A fluorescent label or an enzyme tag, such as urease, alkaline phosphatase or peroxidase, instead of radioactive or other environmental undesirable reagents can also be used. In the case of enzyme tags, colorimetric indicator substrates are known which can be employed to provide a means visible to the human eye or spectrophotometrically, to identify specific hybridization with complementary nucleic acid-containing samples.

Hybridization reactions can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, Sambrook, et al. (1989) supra.

One can also utilize detect and quantify mRNA level or its expression using quantitative PCR or high throughput analysis such as Serial Analysis of Gene Expression (SAGE) as described in Velculescu, V. et al. (1995) Science 270:484-487. Briefly, the method comprises isolating multiple mRNAs from cell or tissue samples suspected of containing the transcript. Optionally, the gene transcripts can be converted to cDNA. A sampling of the gene transcripts are subjected to sequence-specific analysis and quantified.

General procedures for PCR are taught in MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides.

After amplification, the resulting DNA fragments can be detected by agarose gel electrophoresis followed by visualization with ethidium bromide staining and ultraviolet illumination. A specific amplification of differentially expressed genes of interest can be verified by demonstrating that the amplified DNA fragment has the predicted size, exhibits the predicated restriction digestion pattern, and/or hybridizes to the correct cloned DNA sequence.

Probes also can be attached to a solid support for use in high throughput screening assays using methods known in the art. International PCT Application No. WO 97/10365 and U.S. Pat. Nos. 5,405,783, 5,412,087 and 5,445,934, for example, disclose the construction of high density oligonucleotide chips which can contain one or more sequences. The chips can be synthesized on a derivatized glass surface using the methods disclosed in U.S. Pat. Nos. 5,405,783; 5,412,087 and 5,445,934. Photoprotected nucleoside phosphoramidites can be coupled to the glass surface, selectively deprotected by photolysis through a photolithographic mask, and reacted with a second protected nucleoside phosphoramidite. The coupling/deprotection process is repeated until the desired probe is complete.

The expression level of a gene can also be determined through exposure of a sample suspected of containing the polynucleotide (e.g., serum) to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See, U.S. Pat. Nos. 5,578,832 and 5,631,734. The obtained measurement is directly correlated with gene expression level.

The probes and high density oligonucleotide probe arrays also provide an effective means of monitoring expression of a multiplicity of genes, one of which includes an mRNA encoding PKM2. Thus, the expression monitoring methods can be used in a wide variety of circumstances including detection of ovarian cancer, identification of differential gene expression between samples isolated from the same patient over a time course, or screening for compositions that upregulate or downregulate the expression of the M2 isoform at one time, or alternatively, over a period of time.

Alternatively, a label may be added directly to the original nucleic acid sample (e.g., mRNA, polyA, mRNA, cDNA, etc.) or to the amplification product after the amplification is completed. Means of attaching labels to nucleic acids are known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²p) enzymes (e.g., horseradish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. Thus, for example, radiolabels may be detected using photographic film or scintillation counters, fluorescent markers can be detected using a photodetector to detect emitted light. Enzymatic labels are typically detected by providing the enzyme with a substrate and detecting the reaction product produced by the action of tenzyme on the substrate, and colorimetric labels are detected by simply visualizing the colored label.

Patent Publication WO 97/10365 describes methods for adding the label to the target (sample) nucleic acid(s) prior to or alternatively, after the hybridization. These are detectable labels that are directly attached to or incorporated into the target (sample) nucleic acid prior to hybridization. In contrast, “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids, see LABORATORY TECHNIQUES IN BIOCHEMISTRY AND MOLECULAR BIOLOGY, Vol. 24: Hybridization with Nucleic Acid Probes, P. Tijssen, ed. Elsevier, N.Y. (1993).

The nucleic acid sample also may be modified prior to hybridization to the high density probe array in order to reduce sample complexity thereby decreasing background signal and improving sensitivity of the measurement using the methods disclosed in International PCT Application No. WO 97/10365.

Results from the chip assay are typically analyzed using a computer software program. See, for example, EP 0717 113 A2 and WO 95/20681. The hybridization data is read into the program, which calculates the expression level of the targeted gene(s). This figure is compared against existing data sets of gene expression levels for diseased and healthy individuals. A correlation between the obtained data and that of a set of diseased individuals indicates the onset of a disease in the subject patient. The following examples are intended to illustrate and not limit the invention.

Experimental Examples

ELISA Assay

The commercially available Tumor M2-PK ELISA test kit was purchased from SHEBO Biotech (SCHEBO Biotech Tumor M2-PK In- Vitro diagnostic test, Catalog # 08). This is a 96-well formatted test ELISA contains PKM2 control solutions that range from 5 to 100 PKM2 units / ml of plasma. A positive control sample for PKM2 is also included in the kit that is specific to a reference concentration of 15 units/ml which the manufacturer claims corresponds to a specificity of 90% for a control group with diseases other than tumors (n=393). Restated, this reference standard of 15 units/ml plasma, serves as a threshold indicator of tumor burden.

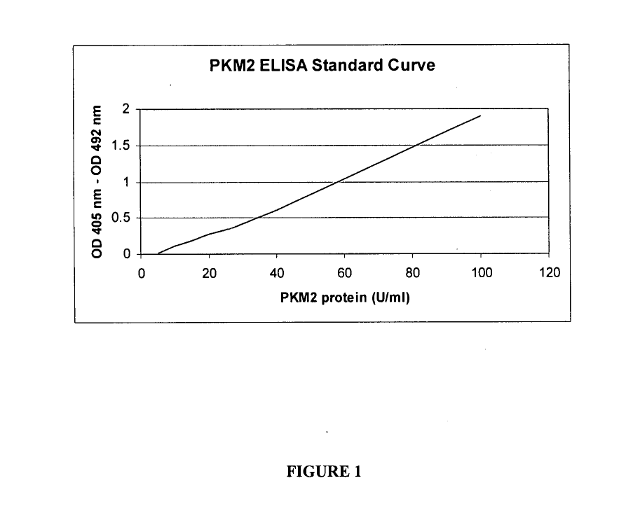

The manufacturer states that the sale of the Tumor M2-PK ELISA test kit is for in vitro diagnostic use only. The manufacturer also states that the concentration of patient samples (diluted to 1:100), can be read directly from the standard curve since the dilution factor (1:100), was taken into account in the production of the standards. See FIG. 1.

All reagents were brought to room temperature. Each patient serum sample was diluted in Phosphate Buffered Saline (PBS) / 5% non-fat dry milk. Biotin labeled was prepared in anti-tumor M2-PK secondary antibody solution (60 μl stock Antibody plus 6.0 ml 1× SCHEBO ELISA wash buffer.) 50 μl PKM2 standards was pipetted into top row of 96-well test plate at the following concentrations.

Standard 1=5.0 U/ml

Standard 2=15.0 U/ml

Standard 3=40.0 U/ml

Standard 4=100.0 U/ml

50 μl of 1:100 diluted test serum samples was then pipetted in duplicate. The samples were incubated for 60 minutes at room temperature. The wells were emptied and the entire plate was washed 3× with 250 μl 1× wash buffer/well. 50 μl/well of the 1:100 biotin-conjugated secondary monoclonal antibody was added to the wells and incubated a further 30 minutes at room temperature.

The wells were emptied and the entire plate was washed 3× with 250 μl 1× wash buffer/well. 50 μl/well stock POD-Streptavidin reagent was added and the combination was allowed to incubate 30 minutes at room temperature in the dark.

The wells were emptied and the entire plate was washed 3× with 250 μl 1× wash buffer/well. 100 μl/well of the stock POD Substrate Solution was added per well and the plate was allowed to incubate 30 minutes at room temperature in the dark. The reaction was stopped by adding 100 μl/well 0.5 M·H₂SO₄

The plate was read at 405 nm with a reference wavelength at 492 nm. The following Table summarizes the results of this ELISA analysis.

TABLE 1
PKM2 Serum Protein Levels by ELISA

| | | | Total # |
|---|---|---|---|
| | Total # of Serum | Total # Samples | Samples |
| | Samples analyzed | above 20 Units/ml | above 100 |
| | by ELISA | Tumor Threshold | units/ml |
| Normal Human | 84 | 11 (13%) | 1 (1%) |
| Donor Serum | | | |
| Ovarian Cancer | 83 | 72 (87%) | 50 (60%) |
| Patient Serum | | | |
| Normal Pregnant | 72 | 42 (58%) | 19 (26%) |
| Donor Serum | | | |

ELISA analysis on 83 ovarian cancer patient serum samples revealed several important discoveries. Based on a simple percentage, 72 out of 83 ovarian cancer patient serum samples (86.7% of the 83 patient test set) contained PKM2 protein levels higher than the tumor burden threshold value for the PKM2 positive control sample which is at 20 Units/mi. Of those 72 samples which tested positive for elevated PKM2 protein, 50 (60% of the 83 patient test set) had a PKM2 protein level of greater than 100 Units/ml. And of these 50 samples, 35 (42% of the 83 patient test set) were patients with stage III or worse ovarian cancer.

This data is in stark contrast to the ELISA analysis of normal human donor serum samples that were tested for PKM2 protein levels. In this case, based on a simple percentage, 11 out of 84 normal donors (13% of the 84 normal donor set) contained PKM2 protein levels higher than the tumor burden threshold value for the PKM2 positive control sample which is at 20 Units/ml. Of those 11 samples which tested positive for elevated PKM2 protein, only one normal donor had a PKM2 serum protein level at 1.1 fold over 100 Units/ml.

Interestingly, when a set of presumed normal human serum was analyzed by ELISA, (this was all serum from ‘normal pregnant females’), the PKM2 levels were as follows. Based on a simple percentage, 42 out of 72 normal pregnant female donors (58% of the 72 person donor set) contained PKM2 protein levels higher than the tumor burden threshold value for the PKM2 positive control sample which is at 20 Units/ml. Of those 42 samples which tested positive for elevated PKM2 protein, 19 (26%) of the normal pregnant female donors had a PKM2 serum protein level at >1 fold over 100 Units/ml.

PKM2 Western Analysis

For Western analysis, a COS cell lysate was prepared from cells that had been transiently transfected with a plasmid containing the PKM2 expression cassette. The protocol is as follows.

NuPAGE Novex Bis-Tris 4-12% Acrylamide Gradient Gels were run using Invitrogen's XCell SureLock Mini-Cell Electrophoresis apparatus. Preparative gels 1.0 mm×2D well format were used with molecular weight ladder loaded in the offset well.

The samples for electrophoresis were prepared as follows:

Sample 10 μg/single lane or 150 μg for the preparative scale well.

NuPAGE LDS sample buffer (4×)

NuPAGE reducing agent (10×)

Samples were heated to 70° C. for 10 minutes before loading.

Each electrophoresis chamber was filled with 1× NuPAGE MOPS SDS running buffer. The upper buffer chamber was filled with 1× NuPAGE MOPS SDS buffer containing 0.25% antioxidant reagent. The gels were run at constant voltage 200V for 60 minutes (100-125 mA/gel start; 60-80 mA/gel end).

The gel was transferred to nitrocellulose filter using tris-glycine MeOH transfer buffer 100V for 90 minutes in a Hoeffer trans-blot apparatus and the filter was rinsed briefly in water, allowed to air dry.

A pre-hybridized filter was soaked in in PBS with 5% non-fat dry milk. The filter was then placed into the BioRad Protean trans-blot apparatus and individual lanes were filled with patient serum samples and control mouse monoclonal antibody in the appropriate wells. All hybridizations with serum were at 1:500 dilution in PBS with 5% non-fat dry milk and incubated overnight at 4° C. with slow movement on a platform shaker. The control monoclonal anti-PKM2 antibody (SCHEBO Biotech, Germany), was used at 1:1000.

Filters were removed from the hybridization block and washed 3× with PBS. Washed filters were then incubated with horseradish peroxidase conjugated secondary antibody solution containing PBS and both goat anti-mouse (H+L) and goat anti-human (IgG+IgM) (Jackson Immuno Research Labs), at 1:10,000 dilution.

Filters were again washed 3× with PBS and then incubatee in Western Lightning Chemiluminescent Reagent (Perkin Elmer) for about 1 minute at room temperature. The filters were then wrapped in plastic wrap and then exposed to film for about 5 seconds to about 5 minutes.

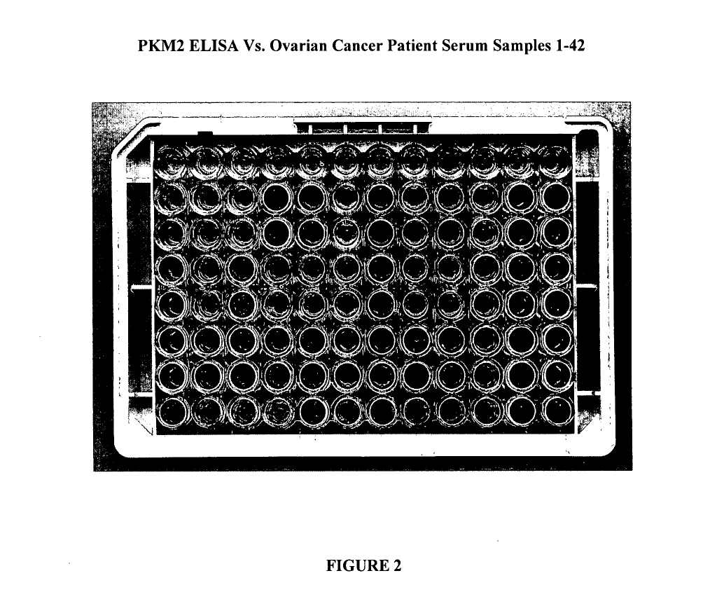

This approach allowed for 11 patient serum samples to be incubated with a single filter. Control hybridizations were set up for each filter. The outside flanking wells were incubated with both a monoclonal antibody specific for human PKM2 (SCHEBO Biotech, Germany) as a positive control, and normal human serum as a negative control. The results are shown in FIG. 2.

Of the 87 ovarian cancer patient serum samples analyzed by Western blot, 14 out of 87 (16%) were scored as positive for exhibiting a PKM2 immune response. This is a subjective assignment based on there being a band on a gel in a lane specific to the patient that was co-localized with the band/hybridization signal emanating from the control monoclonal antibody. 13 out of the 14 patients that were scored as having an immune response to the PKM2 isoenzyme were diagnosed as having stage III or worse cancer, and 12 out of 13 that were screened for PKM2 serum protein showed elevated serum protein levels.

The preceding discussion and examples are intended merely to illustrate the art. As is apparent to one of skill in the art, various modifications can be made to the above without departing from the spirit and scope of this invention.

What is claimed is:

1. A method for diagnosing or prognosing ovarian cancer, comprising detecting the level of expression of pyruvate kinase isoenzyme M2 or fragment thereof in a subject, wherein an overexpression of the isoenzyme is a positive diagnosis or prognosis of ovarian cancer in the subject.

2. The method of claim 1, wherein the level of expression of pyruvate kinase isoenzyme or fragment thereof is determined immunochemically.

3. The method of claim 2, wherein the level of expression of pyruvate kinase M2 isoenzyme or fragment thereof is determined immunochemically by an anti-pyruvate kinase M2 isoenzyme antibody and is substantially non-reacitve with any other pyruvate kinase isoenzyme.

4. The method of claim 1, wherein the level of expression of pyruvate kinase M2 isoenzyme or fragment thereof is determined immunochemically by a anti-pyruvate kinase M2 isoenzyme specific monoclonal antibody, variant or derivative thereof.

5. The method of claim 1, wherein the level of expression of pyruvate kinase M2 isoenzyme or fragment thereof is determined by an antibody selected from the group consisting of a monoclonal antibody, a polyclonal antibody, an antibody variant, an antibody derivative, a humanized antibody and an antibody fragment.

6. The method of claim 1, wherein the level of expression of pyruvate kinase is determed by detecting the amount of pyruvate kinase M2 isoenzyme polynucleotide.

7. The method of claim 1, wherein the detecting is performed on a suitable sample isolated from the subject.

8. The method of claim 7, wherein the suitable sample is preserved tissue sample, an ovarian tissue sample or a sample of body fluid.

9. The method of claim 7, wherein the suitable sample is a sample of body fluid selected from the group consisting of urine, blood and serum.

10. The method of claim 6, wherein the polynucleotide is selected from the group consisting of mRNA and cDNA.

11. The method of any one of claims 1 to 10, wherein the subject is a non-pregnant mammal.
